(19) United States
(12) Patent Application Publication
(10) Pub. No.: US 2026/0335356 A1
(43) Pub. Date: Oct. 29, 2026

(54) TUMOR AND CANCER TREATMENT DEVICES, SYSTEMS AND METHODS

(71) Applicant: Thixomat, Inc., Livonia, MI (US)

(72) Inventors: Raymond F. DECKER, Ann Arbor, MI (US); Anthony MELKENT, Germantown, TN (US); Stephen LEBEAU, Northville, MI (US); Robert CARNAHAN, Park City, UT (US); Steven GOLDSTEIN, Ann Arbor, MI (US); Kurt RIEGGER, Ann Arbor, MI (US)

(21) Appl. No.: 19/116,949

(22) PCT Filed: Oct. 2, 2023
(86) PCT No.: PCT/US2023/034289

§ 371 Date: Mar. 28, 2025

Related U.S. Application Data
(60) Provisional application No. 63/412,259, filed on Sep. 30, 2022.

(51) Int. Cl. A61K 33/06; A61K 9/00; A61K 33/243; A61K 33/30; A61K 33/32; A61K 41/00; A61K 49/04; A61K 51/12

(52) U.S. Cl. CPC A61K 33/06; A61K 9/0024; A61K 33/243; A61K 33/30; A61K 33/32; A61K 41/00; A61K 49/04; A61K 51/1282; A61K 2121/00

(57) ABSTRACT

A Mg—Zn—Ca—Mn alloy for use in the treatment of a malignant tumor, use of a Mg—Zn—Ca—Mn alloy in the manufacture of a medicament as a treatment for a malignant tumor, and a method of treating a malignant tumor comprising administering to a patient a medicament with a Mg—Zn—Ca—Mn alloy. The Mg—Zn—Ca—Mn alloy may consist of 0.3 to 2.0 percent Zn, 0.2 to 1.0 percent Ca and 0.2 to 1.0 percent Mn, the remainder being Mg and inevitable impurities.

### BACKGROUND

### 1. Field of the Invention

The present invention relates to tumor and cancer treatment devices, systems and methods.

### DESCRIPTION OF RELATED TECHNOLOGY

As recently as 2018, estimates for the worldwide occurrence of cancer were at 18,000,000 new cancer cases/year and estimates for the worldwide occurrence of cancer deaths were at 10,000,000 cancer deaths/year. It has also been forecasted, by The John Stoddard Cancer Institute, that 2 in 5 adults will develop cancer by age 75.

Despite many advances in the treatment of cancer, problems with state-of-the-art cancer treatments remain. These include, among others, painful and costly major surgery that may leave the patient disfigured; chemotherapy treatment and the debilitative effects thereof; radiotherapy and damage to surrounding tissue thereby; high death rates, of up to 50%, in some types of cancer; and new cases growing at greater than 10% per year.

Magnesium (Mg) is the lightest of structural metals, at 60% of the density of aluminum (Al), 38% of Ti and 20% of stainless steel or cobalt (Co) implants. Furthermore, the fracture toughness of Mg alloys exceeds that of ceramics, hydroxyapatite, polymers and ceramic implants. It is also important to note that Mg is friendly to the body. Mg is naturally found in bone tissue and is essential to human metabolism. It is also the 4^{th} most abundant cation in the human body, is a co-factor for many enzymes, and stabilizes both DNA and RNA.

It has been reported that, generally, a higher intake of Mg appears to be associated with a reduced risk of colorectal cancer, particularly colon cancer. Benefits have also been claimed in gall bladder and ovarian cases, the latter being greater than with titanium (Ti). It has also been reported that Mg²⁺ inhibits the ovarian SKOV3 cells, and that Mg promotes degradation of Snail 1 protein into the cell nucleus.

A number of reported studies have demonstrated the anti-tumor capabilities of Mg. These studies have been performed in vitro, on cells such as U2OS, SKOV3, LNCaP, MDA-MB-231, MG63, MRMT-1 and MC3T3-E1 cells. But perhaps more importantly, the anti-tumor action of Mg has been performed in vivo. Anti-tumor action of Mg has been recorded in vivo against osteosarcoma (OS), subcutaneous hemangiomas, epithelial ovarian carcinoma (EOC), breast carcinoma, bladder cancer and oral epidermoid cancer.

The benefits of Mg have been related to the byproducts of Mg's bioabsorption, i.e. the release of H₂ and Mg⁺² ions. There is evidence that H₂ gas that is generated by Mg bioabsorption operates as a control on cancer. Additional findings relating to Mg included: in addition to inhibiting inflammation and promoting bone formation, Mg may in combination reduce chemo-drug requirements; tumor cells have an acidic extracellular environment (pH being less than 7.0), while the release of (OH)⁻¹ from Mg alloy implants raises the extracellular pH above 7.0 (while the role of (OH)⁻¹ ions and pH are not fully understood, the benefits of H₂ on humans has been touted); Mg inhibits gall bladder SGC996 cancer cells; H₂ induces tumor cell apoptosis at certain critical concentrations; H₂ decreases reactive oxygen species (ROS) in tumor cells; and tumor growth is inhibited in mice upon release of Zn from biodegrading Mg implanted wires.

A microalloyed Mg—Zn—Ca—Mn alloy, developed by nanoMAG, LLC of Livonia, Michigan, and is formed using a proprietary process to form specific nanostructure in the alloy. The alloy is a bioabsorbable and load bearing and has been used in the form of screws for craniomaxillofacial and other bone implant/fixation applications. This alloy is strong, ductile, biocompatible and bioabsorbable and, therefore, satisfies many orthopedic structural reinforcement requirements during healing—all while matching the strength of non-bioabsorbable titanium (Ti) and exceeding the strength of biodegradable polymeric implants. The biosynthesis of this alloy offers the further important ability to utilize compositional micro-alloying for osteo-promoting bone growth and to serve as a scaffold. The Mg—Zn—Ca—Mn alloy has performed effectively in rabbit femur, canine mandible, canine dental vertical augmentation, sheep ACL and sheep spine trials. Bioabsorption of the alloy has been measured at a rate of 1 mm/yr.

### SUMMARY

The present invention generally relates to the treatment of cancer with magnesium (Mg) alloy, including treatment devices, systems and methods. More specifically, the present invention relates to anti-tumor, bioabsorbable Mg alloy treatment devices, (hereafter just “treatment device(s)”) including implantable devices, for decreasing tumor size and decreasing tumor growth. Even more specifically, the invention relates to a Mg alloy and process that provides localized and targeted anti-tumor control, improved strength, improved ductility, nutrient value for osteo-promotion when necessary, all without toxicity to organs, inflammation or residual particles after bioabsorption. The negative effects of chemotherapy and major surgery thus are obviated. The treatment device is itself a metered, localized source of Mg⁺², Zn, Ca, Mn and H₂ anti-tumor agents.

As such, in one aspect, the invention provides a treatment device, of Mg alloy, that is strong and ductile, providing durability for the entire anti-tumor treatment process.

In another aspect, the treatment device is non-inflammatory and non-toxic to the organs of the body.

In one aspect, the present invention provides a Mg alloy, more specifically a Mg—Zn—Ca—Mn alloy material provided as a treatment device, for treating tumors in the body of a human or animal, the Mg—Zn—Ca—Mn alloy material being microalloyed and consisting of: 0.3 to 2.0 percent by weight of zinc (Zn), 0.2 to 1.0 percent by weight of calcium (Ca), 0.2 to 1.0 percent by weight of manganese (Mn) with the remainder being magnesium (Mg) and inevitable impurities.

In an additional aspect of the invention, the Zn content is in the range of 0.85 to 1.5 percent by weight.

In yet another aspect of the invention the Ca content is in the range of 0.2 to 0.6 percent by weight.

In still a further aspect of the invention, the Mn content is in the range of 0.2 to 0.6 percent by weight.

In an additional aspect of the invention, the Mg alloy includes nanometer-sized ordered zones (mini prisms of 1-3 atom layers) of about 10×0.5 nanometers.

In another aspect, the nanostructures include dislocations, stacking faults, sub-grains, 6H layered crystal structures, α Mn particles and the co-segregation of large and small atoms to these features—on a scale less than 20 nm.

In another aspect, the invention provides for a treatment device, formed of a microalloyed Mg material, for anti-tumor effects and also absorption into the body of a human or animal, the treatment device being formed of Mg microalloyed with Zn, Ca and Mn to form the microalloyed Mg material, with the microalloyed Mg material consisting essentially of 0.3 to 2.0 percent by weight of Zn, 0.2 to 1.0 percent by weight of Ca, 0.2 to 1.0 percent by weight of manganese Mn, with the remainder being Mg and inevitable impurities.

In a further aspect of the invention, the treatment device is one at least one of a wire, a plate, a mesh, a tube, a coil, a foil, powder, a sphere, or a cylinder and is an implantable device.

In another aspect of the invention, the microalloyed Mg material has a yield strength in the range of 200 to 400 MPa, and/or an elongation percentage in the range of 3 to 35 percent, and/or a hardness of 60 to 84 Hv, each of which facilities the use and implantation of the treatment device(s) in thin wire form or segments, or structures, described herein.

In an additional aspect of the invention, the microalloyed magnesium Mg has a grain size of less than 5 μm and a sub-grain size of less than 30 nm.

In another aspect of the invention, the microalloyed Mg material contains Mn particles in the range of 5 to 100 nm diameter.

In yet a further aspect of the invention, the microalloyed Mg material has a H₂ evolution rate of 5 to 30 ml per 21 days in simulated body fluid at 37 °C, thereby allowing the treatment device(s) to provide extended/long term treatment of tumors.

In another aspect of the invention, the treatment devices are inserted percutaneously with an introducer.

In another aspect of the invention, the treatment devices are placed in the void created by the surgical removal of a tumor to treat any residual cancer cells that may remain.

In another aspect of the invention, the treatment devices are placed adjacent to the tumor.

In another aspect of the invention, the treatment devices are placed inside the tumor.

In another aspect of the invention, the treatment devices have voids in them such that the external aspect of the device can displace a certain amount of tissue in the body, but the treatment device contains less the microalloyed Mg material than a solid version of same external volume.

In another aspect of the invention, the treatment devices is in the form of pellets that have a bioabsorbable membrane and contain the microalloyed Mg material.

In another aspect of the invention, the treatment devices are in the form of time release pellets that have multiple bioabsorbable membranes that contain microalloyed Mg material and which release the microalloyed Mg material at different times during the treatment period.

In another aspect, the present invention provides for a method of manufacturing a treatment device, provided as an implantable treatment device, formed at least in part of a material for anti-tumor treatment and absorption into the body of a human or animal, the method comprising the steps of: providing a melt of a Mg material consisting essentially of 0.3 to 2.0 percent by weight of Zn, 0.2 to 1.0 percent by weight of Ca, 0.2 to 1.0 percent by weight of Mn with the remainder being Mg, the melt forming a microalloyed Mg material; forming a product as a casting, squeeze casting, semi-solid metal injection molding or powder from the melt; deforming the product by a thermomechanical deforming process whereby the thickness of the product is reduced an amount greater than 30 percent to form a reduced thickness wrought product; heat treating the reduced thickness wrought product to form a heat treated wrought product and forming the wrought product into an implantable treatment device.

In a further aspect of the invention, the deforming step includes at least one of rolling of the product, extruding of the product; reducing the thickness by greater than 50 percent.

In still a further aspect of the invention, heat treating step includes an annealing step in the range of 200 °C to 400 °C.

In an additional aspect of the invention, annealing step includes annealing for up to 4 hours.

In another aspect of the invention, the hardening step includes age hardening in the range of 150 °C to 225 °C for 10 minutes to 3 hours.

In another aspect, the F factor of processing is in the range or 0.05 to 1.0×10⁶, where F=time (min)×temperature (° K)×% deformation.

In a further aspect of the invention, the forming step forms the product into one of a screw, a plate, a sheet, a foil, a wire, tube, coil powder or cylinder and as an implantable treatment device.

In another aspect, the present invention provides a method of anti-tumor treatment of a patient comprising the steps of: implanting an anti-tumor treatment device in a patient's body whereby the device decreases one or more tumors of the patient, the device being formed of Mg microalloyed with Zn, Ca and Mn to form a microalloyed Mg material, the microalloyed Mg material consisting essentially of 0.3 to 2.0 percent by weight of Zn, 0.2 to 1.0 percent by weight of Ca, 0.2 to 1.0 percent by weight of Mn with the remainder being Mg; attacking the tumor in the patient's body over time as a result of bioabsorption of the treatment device.

In another aspect, the microalloyed Mg treatment device is non-toxic to the body and organs of the body, and does not increase residual elements in blood or urine to a significant level.

In another aspect of the invention, external sources of energy such as x-rays, eddy currents, magnetic or microwave are coupled with the Mg alloy treatment device in order to enhance the anti-tumor effect.

In still a further aspect, the method of treatment with the microalloyed Mg treatment device is used in concert with reduced level of chemotherapy agents.

In another aspect, the microalloyed Mg treatment device is bioabsorbed without leaving intermetallic particles or radioactive particles as residue.

In another aspect, Mn and Zn in solution in the oxide and hydroxide layers of the microalloyed Mg treatment device control the penetration of Cl⁻ and, thus, the release of Mg²⁺ and H₂ to attack the tumor—to match the bioabsorption of the implant to the rate of tumor decrease.

In another aspect, the processed Mg—Zn—Ca—Mn alloy exhibits low texture and fine grain size to contribute to high strength along with high ductility.

In another aspect, coatings are applied to the treatment device to control the initial bioabsorption rate to facilitate the anti-tumor treatment.

In another aspect, a platinum coating is applied to the treatment device and activates a local chemotherapy therapy.

In another aspect, targeting of tumors is optimized to maximize the local availability of Mg²⁺ and H₂, by one or more of the following: targeted spacing of the treatment devices relative to the tumor(s); defining the corrosion rate of the treatment devices; providing protective coatings on the treatment devices; providing cathodic coatings on the treatment devices; pre-charging the treatment devices with H₂ in solid solution; defining a specified geometry of the treatment devices; and defining a specified surface area of the treatment devices.

In another aspect, monitoring devices for the treatment devices during and after surgery is afforded by coating the treatment devices with x-ray indicators, such as platinum (Pt).

In another aspect, the effectiveness of chemotherapy agents is maximized and side effect damage is minimized by targeted partnership with the treatment devices. An example is cisplatin, (a chemotherapy drug, an alkylating agent, containing the metal platinum that damages the DNA of dividing cells in a way that cannot be repaired) wherein Mg can block the kidney damage often associated with that chemotherapy agent. Through its localization at the tumor site, the treatment device loaded with chemotherapy agent may reduce the total dosage and harmful side effect of those agents.

In another aspect, treatment devices are provided to inhibit angiogenesis and metastasis of cancer tumors.

In another aspect, insertion devices are designed for minimum surgical damage, minimum time in surgery and hospital and minimum cost of surgery. One insertion device design is a hypodermic needle type, with single or multiple needles or successive providing of multiple treatment devices, such as wires, in one needle. For skin cancer, in one aspect the treatment device is an adhesive bandage-type plate with multitudinous skin-penetrating needle shaped projections on the skin side.

In another aspect, the treatment devices are implanted using singular or dual Laparoscopes to image the tumor and surrounding tissue, minimizing the surgery needed for typical biopsy diagnostics by combining the imaging methods.

In another aspect, the treatment device is used to introduce tumor treatments, such as radioactive seeds.

In another aspect, the treatment device is placed in vivo using a delivery device that holds a reservoir of the treatment devices to be administered easily by the health care provider.

In another aspect, the delivery device contains a spool of wire as the microalloyed treatment device and which is fed into the delivery device as the health care provider delivers the treatment devices into and/or around the tumor.

In other aspect, the delivery device contains pre-loaded treatment devices that can be easily delivered into the tumor.

In other aspect, the treatment devices are contained in one or more bioresorbable membranes that contain the treatment devices until they can be placed into or near the tumor.

In other aspect, the treatment device is wire that is coiled around the tumor.

Further objects, features and advantages of this invention will become readily apparent to persons skilled in the art after review of the following description, including the claims, and with reference to the drawings that are appended to and form a part of this specification

### BRIEF DESCRIPTION OF THE DRAWINGS

FIG. 1 is an enlarged view of a Mg—Zn—Ca—Mn alloy wire tied into a knot for purpose of illustrating some of the mechanical properties of the Mg—Zn—Ca—Mn alloy wire.

FIG. 2 is graph illustrating a Mg—Zn—Ca—Mn alloy, BioMg 250™ alloy, having been processed for an intermediate bioabsorption rate in vivo in the human body.

FIG. 3A is scanning electron microscope (SEM) micrograph showing a microalloyed Mg material implant, 52 weeks after implantation, and showing a transition layer and new bone layer about the implant with no residual particles of the implant or microalloyed Mg material the new bone layer.

FIG. 3B is SEM micrograph, similar to FIG. 3A, mapping Ca in the surrounding layers.

FIG. 4 is a μCT scan of a healed canine mandible, after 52 weeks with implanted Mg—Zn—Ca—Mn alloy devices, and illustrating an affected thickness transition layer about the implanted devices.

FIG. 5A is an image of the microstructure of a Mg—Zn—Ca—Mn alloy, BioMg 250™ alloy, and illustrates a Mn particles in the microstructure.

FIG. 5B is an image of the microstructure of a Mg—Zn—Ca—Mn alloy, BioMg 250™ alloy, and illustrates GP zones, of Zn and Ca atoms, in the microstructure.

FIG. 6 is a presentation of texture and grain sizes in Mg—Zn—Ca—Mn alloy, BioMg 250™ alloy.

FIGS. 7A-7D are diagrammatic, cross-sectional illustrations of pellets incorporating the cancer treatment devices in various forms.

FIGS. 8A-8D are diagrammatic, cross-sectional illustrations of time release cancer treatment pellets having concentric layers incorporating therein the treatment devices in various forms.

FIGS. 9A-9D illustrate the treatment devices in various forms being introduced into a tumor or treatment site.

FIGS. 10A-10D illustrate the treatment devices in various forms being introduced about a tumor or treatment site.

FIG. 11 is a cross-sectional drawing of a delivery device for delivering the treatment devices to a tumor or treatment site.

FIGS. 12A and 12B are further embodiments of treatment devices in accordance with the principles of the present invention.

### DETAILED DESCRIPTION

Various surgical and visualizations techniques may be employed in conjunction with the treatment devices presented in following discussion. Radiological imaging may be achieved non-invasively by using 2D X-ray images and multiple image computerized tomography to construct 3D images. This may be further augmented by elemental X-ray emission spectroscopy and histology. In addition, ultrasonic imaging may be used to guide placement of the treatment devices discussed hereafter.

Additionally, various diagnostic techniques yield improved resolution down to the cellular level. These include at least the following: nuclear Magnetic Resonance enabling imaging of radiolucent soft tissue; radiation beam therapy using single photon; positron and electron emission sources for cellular detection; and artificial intelligence software. It will be appreciated that such techniques can be employed in the placement and locating of the treatment devices discussed herein.

Minimally invasive use of laparoscopic imaging and sensing devices afford minimal surgical incisions relative to open surgery. 3D imaging of tumorous soft tissue and the surrounding volume can rapidly diagnose the condition without surgery by utilizing MediSCAPE and avoiding tissue removal for pathological analysis. Real time images can be displayed during surgery to aid proper placement of the treatment devices in accordance herewith. Laparoscopic surgery is complimented by Artificial Intelligence software. Compared to open surgery, costs, hospital time and recovery time are lower.

Thin Mg wire is a common shape for in vivo applications. However, Mg wire has insufficient strength to allow a simple needle-like injection of the wire into a tumor. Because of its insufficient strength, the unalloyed Mg wire will bend away from the tumor during insertion. Additionally, in one study, it was determined that unalloyed Mg wire is absorbed too fast in vivo and, therefore, unsuitable to provide long term treatment and care.

Mg—Zn—Ca—Mn alloy, which is the alloy of the BioMg 250™ alloy, can be produced in wire, tube, bar, plate, foil, mesh, and particulate/powder forms that afford the alloy medicament applications for cancer or malignant tumor treatment employing minimally invasive implantation procedures using the above and other technologies. The alloy of the BioMg 250™ alloy is a microalloyed magnesium (Mg) material consisting of 0.3 to 2.0 percent by weight of zinc (Zn), 0.2 to 1.0 percent by weight of calcium (Ca), 0.2 to 1.0 percent by weight of manganese (Mn) with the remainder being Mg and inevitable impurities. In one variant, the alloy of the BioMg 250™ alloy is a microalloyed Mg material consisting of 0.85 to 1.5 percent by weight of Zn, 0.2 to 0.6 percent by weight of Ca, 0.2 to 0.6 percent by weight of Mn with the remainder being Mg and inevitable impurities.

Following are tables and examples that demonstrate the effectiveness of the above Mg—Zn—Ca—Mn alloy:

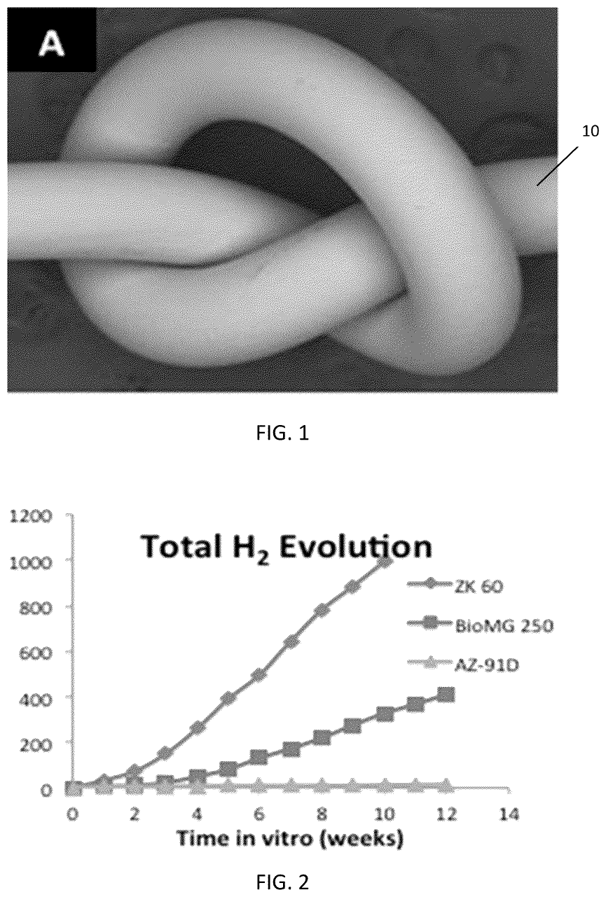

TABLE I
Mechanical Properties of BioMg 250 ™ Wire
(0.3 mm diameter)

| | YS, | UTS, | Elong., | |
|---|---|---|---|---|
| Condition | MPa | MPa | % | Bendability |
| Hard | 400 | 400 | 3 | <1.2 mm radius |
| Anneal C | 352 | 352 | 11 | |
| Anneal D | 228 | 269 | 19 | Tight knot (FIG. 1) |
| | | | | at room temperature |
| | | | | in 0.3 mm diameter |
| | | | | BioMg ™ wire |

Example I. As noted above, unalloyed Mg wire has insufficient strength to allow a simple needle-like injection of the wire into a tumor. Because of its insufficient strength, the unalloyed Mg wire will bend away from the tumor during insertion. However, Mg—Zn—Ca—Mn alloy, and BioMg 250™ in particular, can be processed to afford simple hypodermic needle-like injection to a tumor and/or around tumors, when necessary. Whereas unalloyed Mg wire would have a yield strength less than 200 MPa, the Mg—Zn—Ca—Mn alloy can be processed to levels of up to 400 MPa. Furthermore, unalloyed Mg is notoriously brittle because of its hcp crystal structure, the BioMg 250™ alloy is microalloyed and processed to activate multiple slip systems to enhance ductility and bendability, thus avoiding fracture during insertion. (See Table I above and wire 10 in FIG. 1) With the Mg—Zn—Ca—Mn alloy, yield strength levels of 300 MPa were attained with a) a hot deformation cycle with the factor, F, of 0.5×10⁶, where F=time×temperature×% deformation (thickness reduction). The same strength level was also attained with an F factor of 0.5×10⁶ with a cold deformation cycle. The resulting wire or wire segments 10 exhibit sufficient strength to allow insertion of the wire or wire segments 10 into the tumor itself or tissue around the tumor site.

Example II. Corrosion in Synthetic Body Fluid (SBF) is illustrated in FIG. 2 with BioMg 250™ alloy processed for an intermediate bioabsorption rate in vivo in the human body. The corrosion of BioMg 250™ alloy in SBF is presented in comparison to two other common Mg alloys, ZK60 and AZ91D alloys. Absorption/corrosion is plotted in ml of H₂ vs. weeks. As seen in the FIG. 2, the ZK60 alloy is absorbed/corroded at a rate twice as fast as the BioMg 250™ alloy. Because of its absorption timeline, ZK60 alloy would not be viable for long term treatment options. The AZ91D alloy showed virtually no corrosion/absorption and, for contrasting reasons, would not be viable for long term treatment options. Accordingly, the inventors have discovered that the BioMg 250™ alloy can be specifically targeted for long-term cancer treatment options.

Example III. Toxicity and biocompatibility are major concerns when metals are implanted in the human body. In a study of changes to various organs, 26 weeks after implantation of an implant formed of BioMg 250 ™ alloy in a canine mandible revealed no evidence of adverse pathological changes (e.g. necrosis and/or exuberant inflammation), as a result of exposure to the BioMg 250™ alloy, were found in the brain, heart, kidney, liver, lung with bronchi, mandibular lymph node, spleen and thymus of the canine test subject. The implant dimensions were 2×8×100 mm and the bioabsorption of the implant was at a rate of 0.5 mm/yr. Accordingly, the BioMg 250™ alloy can be implemented in cancer treatment without major concerns regarding adverse effects to other organs of the body.

Example IV. Toxicity testing results of BioMg 250™ alloy is presented in Table II, below. As seen therein, the BioMg 250™ alloy exhibited no risk, no mutagenesis and mild reactivity. Accordingly, BioMg 250™ alloy does not presenting a toxicity risk to a patient during cancer treatment.

TABLE II
ISO 10993 Testing of BioMg 250 ™ Toxicity/Safety

| | ISO | |
|---|---|---|
| Test Description | Standard | Results |
| Extractables & Leachables | 10993-12 | Extractables pose no |
| with Toxicology | | risk to patients at |
| Assessment | | levels detected. |
| Cytotoxicity - ISO Elution | 10993-5 | Mild reactivity - |
| | | Grade 2 |
| Systemic Toxicity Extracts | 10993-11 | Extracts met |
| in Mouse Model | | requirement with |
| | | proper extraction |
| | | techniques |
| Sensitization in Guinea | 10993-10 | No evidence of |
| Pigs | | sensitization |
| Intracutaneous Reactivity | 10993-10 | Met requirements |
| in Rabbits | | of test |
| Genotoxicity - Ames | 10993-3 | Extracts were |
| Assay | | non-mutagenic |
| Genotoxicity - in Vitro | 10993-3 | Test article was not |
| Mouse Lymphoma | | mutagenic |
| Assay | | |

Example V. Urine tests were taken during 104 weeks in the canine mandible study mentioned above and the results are presented below in Tables Ill and IV. While some changes in calcium (Ca) levels were observed, the changes were not clinically significant and Mg is exported in urine. While Ca does show statistical differences at weeks 13 and 104, the differences are not clinically significant. All other comparisons lack statistical significance.

TABLE III
P-Values for Two-sample T-tests performed on Urine
ICP-MS results

| | P-values for Two-Sample T-Tests | | | |
|---|---|---|---|---|
| | Magnesium | Calcium | Zinc | Manganese |
| Pre-op vs | 0.244 | 0.683 | 0.657 | 0.286 |
| Week 4 | | | | |
| Pre-op vs | 0.260 | 0.047 | 0.726 | 0.093 |
| Week 13 | | | | |
| Pre-op vs | 0.053 | 0.281 | 0.791 | 0.818 |
| Week 26 | | | | |
| Pre-op vs | 0.076 | 0.222 | 0.546 | 0.226 |
| Week 39 | | | | |
| Pre-op vs | 0.144 | 0.360 | 0.433 | 0.985 |
| Week 52 | | | | |
| Pre-op vs | 0.111 | 0.037 | 0.487 | 0.653 |
| Week 104 | | | | |

Analysis does not assume equal variances

TABLE IV
Averages for the Urine ICP-MS results for each Metal
Ion at each Time Point

| | Urine Results Averages | | | |
|---|---|---|---|---|
| Week | Magnesium | Calcium | Zinc | Manganese |
| 0 | 8.11 | 7.98 | 0.1016 | 0.0073 |
| 4 | 11.65 | 7.36 | 0.0880 | 0.0062 |
| 13 | 14.45 | 11.13 | 0.1342 | 0.0047 |
| 26 | 17.14 | 10.25 | 0.1122 | 0.0069 |
| 39 | 18.18 | 9.78 | 0.1276 | 0.0056 |
| 52 | 22.82 | 12.03 | 0.1373 | 0.0074 |
| 104 | 27.01 | 15.17 | 0.1352 | 0.0091 |

All results are listed in parts per million

Example VI. During the 104 week canine mandible study of BioMg 250™ implants mentioned above, blood tests were performed to test for the absorption of the alloying elements. As seen in Tables V and VI, the results of the blood tests showed no significant increase in Mg, Zn, Ca and Mn levels. Accordingly, no differences were concluded at 95% confidence.

TABLE V
P-values for Two-Sample T-tests performed on Blood
Serum ICP-MS Results

| | P-values for Two-Sample T-Tests | | | |
|---|---|---|---|---|
| | Magnesium | Calcium | Zinc | Manganese |
| Pre-op vs | 0.903 | 0.979 | 0.577 | 0.630 |
| Week 4 | | | | |
| Pre-op vs | 0.681 | 0.855 | 0.854 | 0.723 |
| Week 13 | | | | |
| Pre-op vs | 0.864 | 0.975 | 0.588 | 0.389 |
| Week 26 | | | | |
| Pre-op vs | 0.786 | 0.194 | 0.435 | 0.311 |
| Week 39 | | | | |
| Pre-op vs | 0.932 | 0.223 | 0.836 | 0.262 |
| Week 52 | | | | |
| Pre-op vs | 0.918 | 0.808 | 0.945 | 0.714 |
| Week 104 | | | | |

Analysis does not assume equal variances

TABLE V
Averages for the Blood Serum IC

| | Blood Serum Results Averages | | | |
|---|---|---|---|---|
| Week | Magnesium | Calcium | Zinc | Manganese |
| 0 | 37.50 | 47.01 | 3.318 | 0.0236 |
| 4 | 37.26 | 46.96 | 3.469 | 0.0217 |
| 13 | 36.78 | 47.31 | 3.362 | 0.0250 |
| 26 | 37.20 | 46.95 | 3.465 | 0.0204 |
| 39 | 37.00 | 44.97 | 3.604 | 0.0279 |
| 52 | 37.66 | 45.03 | 3.387 | 0.0283 |
| 104 | 37.67 | 46.57 | 3.295 | 0.0253 |

All results are listed in parts per million
P-MS Results for Each Metal Ion at Each Time

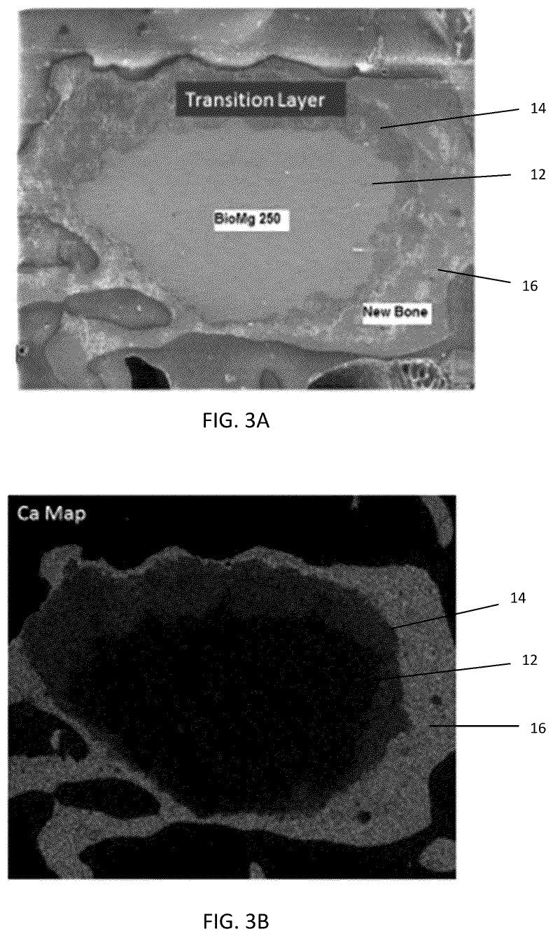

Example VII. In another study, a BioMg 250™ alloy device 12 was implanted for 52 weeks in the femur of a New Zealand rabbit. As seen in the SEM micrograph of FIG. 3A, in the transition and new bone layers 14, 16 around the bioabsorbing implant 12, there are no residual alloy particles. Also, the implant 12 is seen to be osteo-promotive, as evidenced in the SEM map of Ca, which is presented in FIG. 3B. In the transition layer, Mn at 0.2 to 0.5% and Zn at 1.0 to 1.7% were detected. The presence and percentages of Mn and Zn in the alloy are utilized to control rate of corrosion and bioabsorption of the alloy. From this, the bioabsorption rate of the present treatment device can be matched to the desired rate of Mg²⁺ and H₂ release for desired the anti-tumor effect of the treatment device. More specifically, the Mn and Zn in solution in the oxide and hydroxide layers of the treatment device control the penetration of Cl⁻ into the treatment device. Thus, variations in Mn and Zn may be used to control the release of Mg²⁺ and H₂ to attack the tumor, so as to match the desired bioabsorption of the implant to desired rate of tumor decrease.

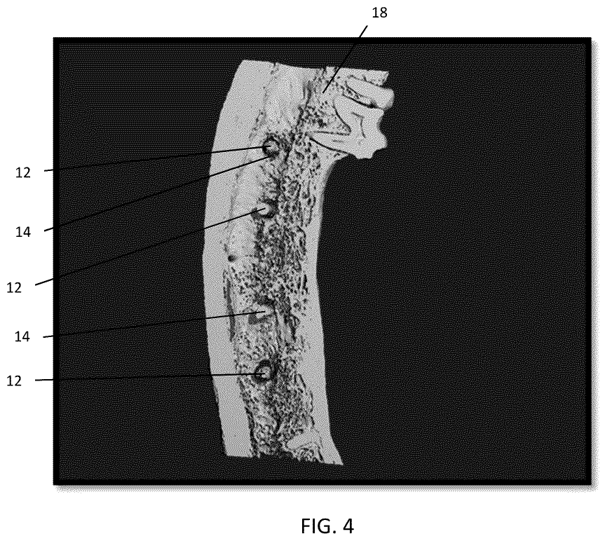

Example VIII. In the long-term canine mandible studies mentioned above, a μCT scan of a healed canine mandible 18 after 52 weeks, with implanted BioMg 250™ alloy devices 12, was taken and is presented in FIG. 4. The μCT scan illustrated that the affected thickness of the transition layer 14 about each of the implanted BioMg 250™ alloy devices, which were screws, was about 1 mm.

The spacing of individual Mg—Zn—Ca—Mn alloy wires, when placed in an array on a tumor, should be about 2 mm, representing a 1 mm affected area for each of the two adjacent wires.

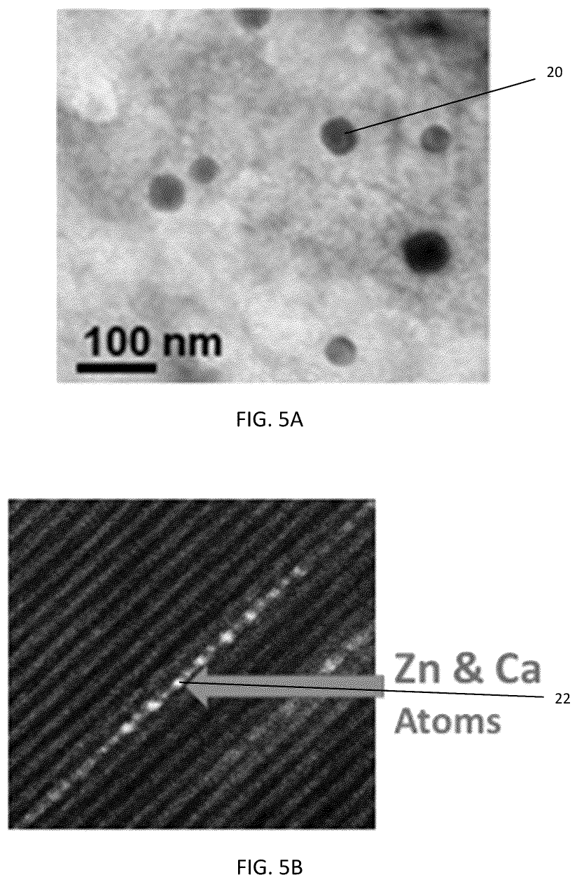

Example IX. The nanostructure of the BioMg 250™ alloy is unique and is a result of its particular processing. One of the microalloying elements is a large atom element having an atomic size larger than the atomic size of a Mg atom and another of the microalloying elements is a small atom element having an atomic size smaller than the atomic size of the Mg atom. The atoms of the large atom element, which is Ca, have atomic radii of 173 Angstroms or more, and atoms of the small atom element, which is at least one of Zn and Mn, have atomic radii of 145 Angstroms or less. As a result, the microalloying elements have an atomic diameter 10% larger or 10% smaller than the Mg atoms of the alloy. The Ca atoms also have an electronegativity of 1.1 or less and the Zn and Mn atoms have an electronegativity of 1.4 or more, which is 10% less and 10% greater than the Mg atoms of the alloy. As a result of novel processing, strengthening and ductilizing results from clusters or short range ordered zones (known as Guinier-Preston (GP) zones) that form on the basal {0001}planes of Mg. Two features of the nanostructure are illustrated in FIGS. 5A and 5B, which respectively show a Mn particles 20 and GP zones 22 (providing strength). The a Mn particles were of a fine grain size and in the range of 8 to 120 nm. The GP zones are decorated by/exhibit co-segregated atoms of Ca and Zn, and were accordingly of 0.5 to 15 nm. Furthermore, additional special<20 nm features have been identified by high definition electron microscopy; namely, stacking faults, <c+a> dislocations, sub-grains, 6H layered crystal structures—with Zn/Ca segregated to each of these features. Intermetallic particles of >1 μm are minimized, while continuous films of these intermetallics and resultant denuded grain boundaries are avoided, resulting in high strength and ductility.

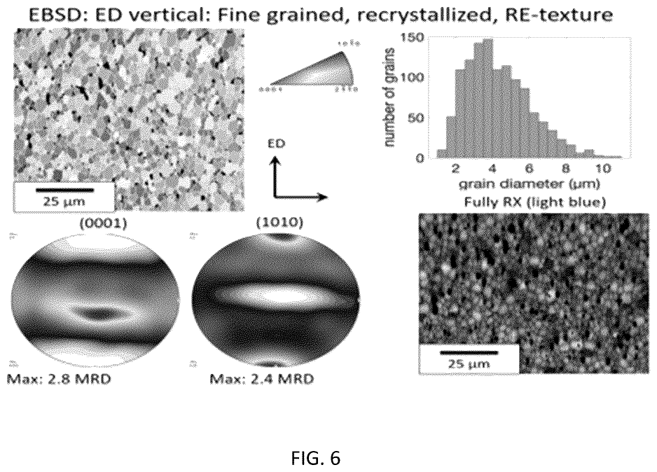

Example X. The low texture, MRD 2.4 to MRD 2.8, in the BioMg 250™ alloy (presented in FIG. 6) makes for high ductility. This compares with MRD of >10 for brittle unalloyed Mg. The fine grain size, also presented in FIG. 6, boosts strength and ductility of the treatment devices.

Example XI. The effect of Mg—Zn—Ca—Mn alloy, specifically BioMg 250™ alloy wire, as a medicament or treatment device on lung cancer cells and healthy cells were evaluated using an ex vivo “sandwich” test on mouse lung tissue. BioMg 250™ alloy wire, of 0.3 mm diameter, was placed adjacent to both healthy cells and A549 cancer cells in the sandwich composed of mouse lung slices. The BioMg 250™ alloy wire bioabsorbed from 0.3 mm to 0.10 mm diameter in 7 days. High maintenance (>85%) of murine healthy cells was observed; whereas, A549 cancer cell toxicity was seen as being induced nearby the BioMg 250™ alloy wire.

Example XII. BioMg 250™ alloy wire used in the above lung cancer cell tests was evaluated for mechanical properties. The 0.1 mm diameter wire 10 had Yield Strength of 402 MPa, Ultimate Tensile Strength of 467 MPa and Elongation of 5%. The BioMg 250™ alloy wire 10 was wound on a 0.7 mm diameter rod and tied in a tight knot, as seen in FIG. 1, without fracture. The 0.3 mm diameter wire had Yield Strength of 373 MPa, Ultimate Tensile Strength of 413 MPa and Elongation of 4%. The latter wire was wound on 1.2 mm diameter rod without fracture.

Example XIII. The processing of BioMg 250™ alloy stock was established and confirmed to Good Manufacturing Practice standards and nanoMAG's Quality Management Systems standards. These practices were audited to meet FDA standards. Heat Treated 10 mm diameter bar from this stock had a Yield Strength of 287 MPa, Ultimate Tensile Strength of 308 MPa, Elongation of 12% and corrosion rate of 0.44 to 0.63 mm/yr in an SBF solution.

Example XIV. Kirschner wire (also known as K wire or K pins) constructed of BioMAG250™ alloy at diameters of 1.1, 1.6 and 2.0 mm were evaluated for corrosion rates in SBF and determined to have corrosion rates in the range of 0.45 to 0.52 mm/yr therein.

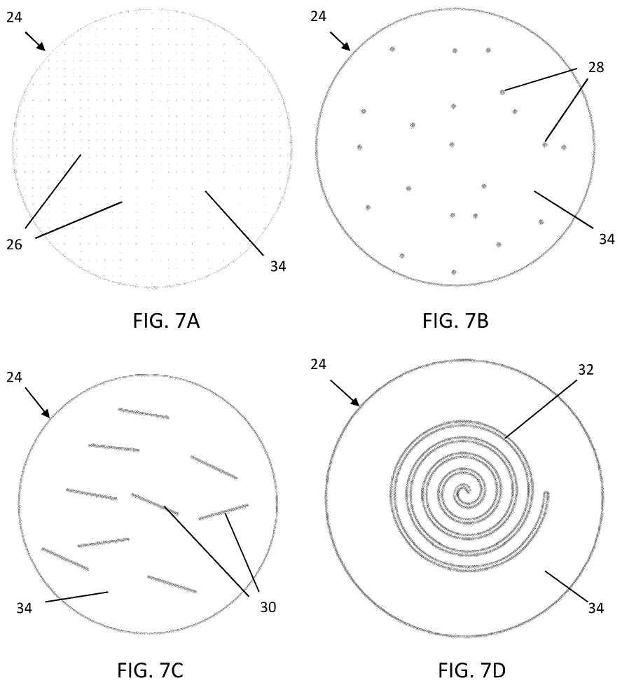

Referring now to FIGS. 7A, 7B, 7C and 7D, illustrated in diagrammatic cross-sections are medicaments 24, also herein referred to as treatment devices 24, presented as single release pellets having Mg—Zn—Ca—Mn alloy or BioMg 250™ alloy materials provided in formats such as powder 26, spheres or particles 28, cut wires 30 (cylinders), and coils 32. The Mg/Mg alloy materials may be suspended in a solid bioresorbable material 34 (such as bioasbsorable polymers) or may be suspended in fluids or gels (such as hydrogel) encased by membrane layers (formed of bioabsorbable polymers).

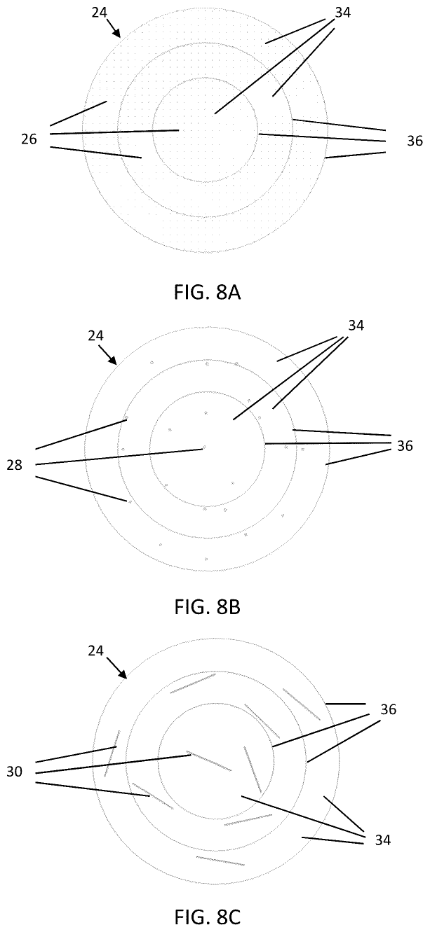

As seen in the diagrammatic cross-sections of FIGS. 8A, 8B and 8C, the treatment devices 24 may be presented as time release pellets provided as concentric rings 36, three concentric rings being illustrated in these figures. The concentric rings 36 allow for a time-release of the therapeutic Mg—Zn—Ca—Mn alloy or BioMg 250™ alloy materials provided in formats such as powder 26, spheres or particles 28, cut wires 30 (cylinders), and coils 32 (not shown). The outer ring or region 36 is released first, followed by next inner region 36, and followed by next inner region 36. The rings 36 may be formed of any of the suspension materials, fluids, gels and membranes 34 utilized in the single release presentation.

The time-release or single release pellets, or other presentations of the treatment devices as just powder 26, spheres 28, cut wires 30 (cylinders) and coils 32, may be placed adjacent to or inside the tumor to provide long term treatment thereof. Alternatively, the treatment devices may be placed in a void created by the surgical removal of the tumor. In this latter case, the pellet(s) (or other treatment device(s)) provide the cancer killing/controlling therapeutic in case residual cancer cells remain after surgical removal of tumor.

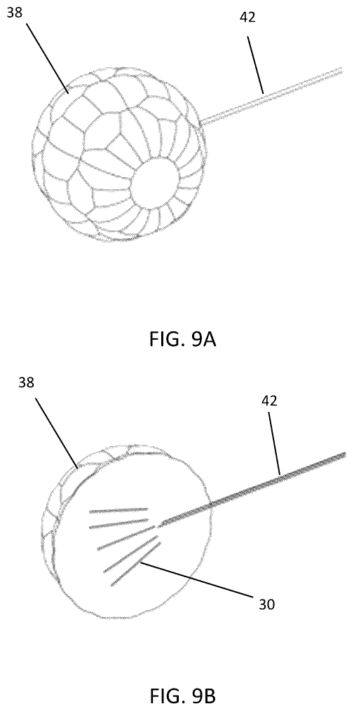

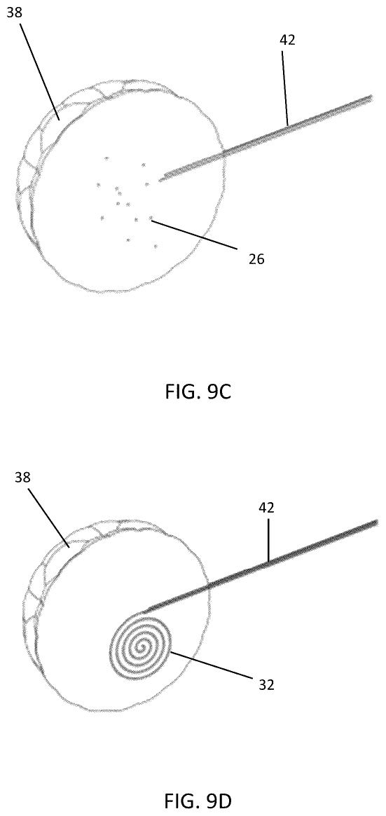

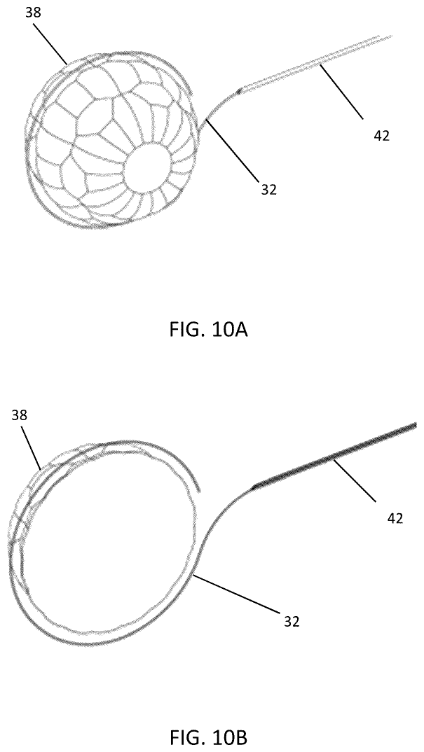

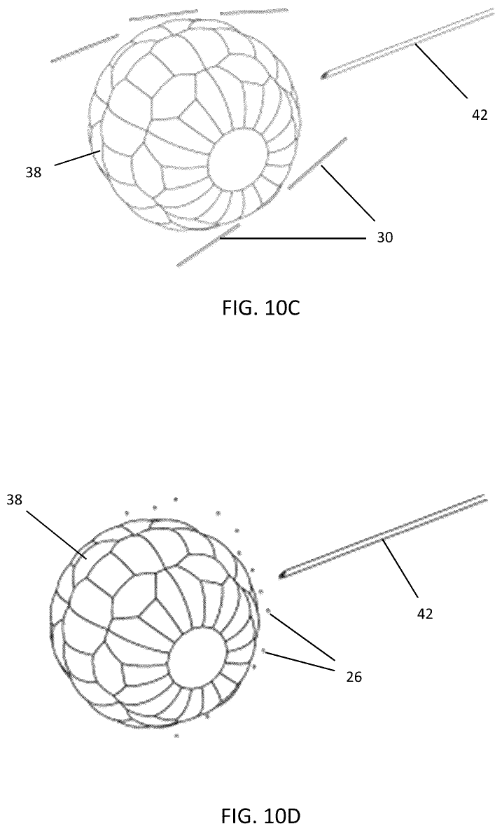

Means and placement of the various forms of the Mg—Zn—Ca—Mn alloy or BioMg 250™ alloy medicaments/treatment devices in tumors 38 is illustrated in FIGS. 9A-9D. Means and placement of the various forms of the Mg—Zn—Ca—Mn alloy or BioMg 250™ alloy treatment devices around tumors is illustrated in FIGS. 10A-10D. These figures show a variety of potential Mg—Zn—Ca—Mn alloy or BioMg 250™ alloy treatment devices, in addition to the pellets of FIGS. 7A-D and 8A-8C. In FIGS. 9A-9D, Mg—Zn—Ca—Mn alloy or BioMg 250™ alloy treatment devices are presented simply as cylindrical (cut wire) segments devices 30 (FIGS. 9B and 10C), spherical or powder devices 26, 28 (FIGS. 9C and 10D) and coiled wire devices 32 (FIGS. 9D and 10B) of Mg—Zn—Ca—Mn alloy or BioMg 250™ alloy material. The coiled wire device is shown as a single coiled bundle, but it will be readily appreciated that it comprise multiple coils or coil bundles within or about the tumor. It will also be appreciated that the Mg—Zn—Ca—Mn alloy or BioMg 250™ alloy treatment devices may be provided in other configurations and shapes beyond powder, spherical, cylindrical and coiled shapes. While the above treatment device(s) may be placed in or near a tumor for treatment, the device(s) may also be placed in a void created by removal of a tumor or in a separate void created by a surgeon. In such instances, the treatment device is used in case any residual cancer or tumor cells remain after the surgical removal.

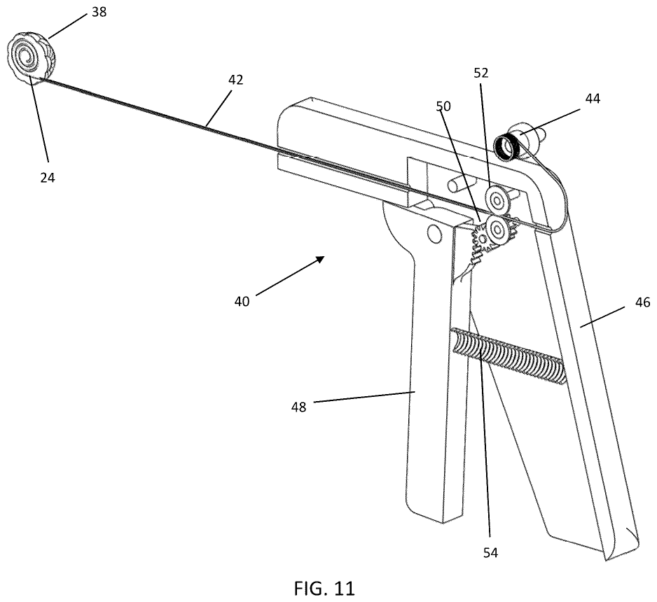

FIG. 11 shows an exemplary delivery device 40, in cross-section, that can be used to deliver the Mg—Zn—Ca—Mn alloy or BioMg 250™ alloy treatment devices. The delivery device 40 is shown in a pistol grip style, but the form can be modified into a number of configurations, such as a syringe style, in-line handle style or other configuration. The delivery device 40 includes, as an introducer 42, a hollow awl with a distal tip that directs and may be used to place the Mg—Zn—Ca—Mn alloy or BioMg 250™ alloy treatment device(s) 24, which are delivered through the introducer 42. The tip may be sharp to allow for easy penetration of the introducer through skin or other soft tissue. Thereafter, the introducer 42 may be positioned about or into tumor 38 and the treatment device(s) 24 dispensed into the tumor 38 or surrounding tissue. Placement of the treatment devices about or in the tumor 38 may be specific, such as in an array at 2 mm intervals, or non-specific with now defined spacing, etc. The tip of the introducer 42 may also have a coiling feature that coils a Mg—Zn—Ca—Mn alloy or BioMg 250™ alloy wire that is delivered through it. Alternatively, the tip may have a directional feature that places the treatment device to the side or at an angle relative to the tip. The delivery device 40 may have a reservoir 44 that holds a bulk amount of Mg—Zn—Ca—Mn alloy or BioMg 250™ alloy treatment devices. The reservoir 44 can also consist of a spool where a bulk amount of Mg—Zn—Ca—Mn alloy or BioMg 250™ alloy treatment device, in wire form, can be stored.

As illustrated, the delivery device 40 has a handle 46 and an actuator (trigger) 48 that delivers the Mg—Zn—Ca—Mn alloy or BioMg 250™ alloy treatment device(s) through the hollow awl to the treatment site. By squeezing the trigger 48 of the delivery device 40, a gear set 50, coupled to the trigger, rotates at least one of a pair of drive wheels, causing the Mg—Zn—Ca—Mn alloy or BioMg 250™ alloy treatment device, located between the drive wheels 52, to be advanced in the hollow awl and out through the tip. As seen in FIG. 11, the trigger 48 is biased by a spring 54 and a one-way clutch, integrated with the gear set 50, allows the trigger 48 to return to its initial position for delivering an additional predetermined amount of the Mg—Zn—Ca—Mn alloy or BioMg 250™ alloy treatment device(s) upon a subsequent squeezing of the trigger 48.

The delivery device 40 may also include a cutting feature, for cutting into discreet segments or cylinders, a Mg—Zn—Ca—Mn alloy or BioMg 250™ alloy treatment device provided in wire form. The delivery device can also be used to coil a Mg—Zn—Ca—Mn alloy or BioMg 250™ alloy treatment device, in the form of wire, around the tumor.

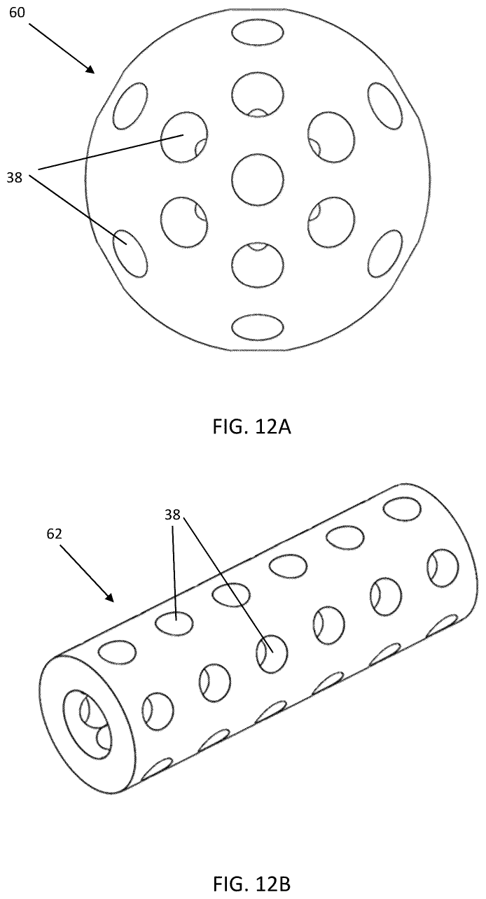

FIGS. 12A and 12B show examples monolithic Mg—Zn—Ca—Mn alloy or BioMg 250™ alloy treatment devices 60, 62. These monolithic bodies are constructed from one or more solid pieces of Mg—Zn—Ca—Mn alloy or BioMg 250™ alloy material so that they can be delivered in bulk and exposed to the tumor or treatment site. The bodies forming the treatment devices 60, 62 may be provided in many possible configurations, including in the form of spheres and cylinders, In the illustrated examples of FIGS. 12A and 12B, the bodies of the treatment devices are provided with holes 64 penetrating into or through the bodies, which also be hollow or solid. Such a construction allow the exterior shape of the treatment device 60, 62 to be large, while the actual volume of Mg—Zn—Ca—Mn alloy or BioMg 250™ alloy forming the treatment device is customized to the desired amount of treatment material to be delivered at the tumor and/or treatment site. These treatment devices 60, 62 can utilize different ways to lessen the bulk of the material used, such as incorporating grooves, dimples, notches, etc., while maintaining a desired external envelope or shape.

The treatment devices, of FIGS. 12A and 12B for example, may be implemented to maximize the effectiveness of chemotherapy agents while minimizing side effect damage, by presenting a targeted partnership with the treatment device(s). As one example, cisplatin, (a chemotherapy drug containing the metal platinum), may be incorporated in powder form into voids of the treatment device or may be provided as a coating on the surfaces, external and/or internal of the treatment device. By varying the available surface area, the amount of amount of associated chemotherapy agent may also be varied. In practice, the chemotherapy agent operates to damage the DNA of dividing cancer cells in a way that cannot be repaired, resulting no further dividing and resultant death of the cells. As a result, the uptake of Mg can aid in blocking the kidney damage often associated with chemotherapy agents. Through its localization at the tumor site, the treatment device operating as a carrier for the chemotherapy agent may reduce the total dosage and harmful side effect of those agents.

In a similar fashion, the treatment devices may incorporate and be used in conjunction with other tumor treatments. For example, the treatment devices may additionally operate as a delivery vehicle for tumor treating radioactive seeds.

In manufacturing the implantable treatment devices, a melt of a Mg—Zn—Ca—Mn alloy material forming a microalloyed Mg material. In one embodiment the melt consisting essentially of 0.3 to 2.0 percent by weight of Zn, 0.2 to 1.0 percent by weight of Ca, 0.2 to 1.0 percent by weight of Mn, with the remainder being Mg and inevitable impurities. In another embodiment, the melt is a Mg—Zn—Ca alloy material forming a microalloyed Mg material. Notably, the absence of Mn may affect the ability of the material to be finely tailored to the desired corrosion rate. From the melt, a product is formed as a casting, squeeze casting, semi-solid metal injection molding. Next the product is deformed by a thermomechanical deforming process whereby the thickness of the product is reduced in an amount greater than 30 percent, preferably greater than 50%, to form a wrought product of reduced thickness. The wrought product is thereafter heat treated to form a final wrought product, which is then formed into or incorporated into the treatment device. Alternatively, the product can be cold worked and deformed by at least one of cold drawing, cold stamping, cold stretching, cold swaging, cold spinning or cold rolling. A process for making a wrought Mg alloy product is disclosed in U.S. application Ser. No. 16/971,579, which is incorporated by reference, in its entirety, herein.

Mg—Zn—Ca—Mn alloy or BioMg 250™ alloy used in the manufacture of a medicament/treatment device, depending on its intended use, may be provided is in the form of one of a wire, cylinder, mesh, sheet, screw, plate, coil, foil, powder or tube. Malignant tumors to which such a medicament/treatment device hereof is applicable includes, without limitation, esophageal, gall bladder, osteosarcoma, bone metastasis, breast, testicular, ovarian, inguinal herniatric, bariatric, sphincter, lung, colorectal, and prostate cancers.

The various disclosed medicaments/treatment devices may also be applied with coatings, such as oxides, to control their initial bioabsorption rate.

Additionally, the various disclosed Mg—Zn—Ca—Mn alloy or BioMg 250™ alloy treatment devices may be provided with cathodic coatings on the treatment devices for the purposes of controlling by absorption.

Further, the various disclosed Mg—Zn—Ca—Mn alloy or BioMg 250™ alloy material of the treatment devices may be pre-charged with H₂ in solid solution, thereby amplifying the presence of H₂ as the treatment device corrodes and is bioabsorbed.

External sources of energy, such as x-rays, eddy currents, magnetic or microwave may also be coupled with the treatment device to enhance the anti-tumor effect, by heating the tumor via the applied energy and the implanted treatment devices.

Monitoring of the implanted treatment devices, after implantation and during treatment, may also be performed. As such the treatment devices may be coated with x-ray indicators, such a platinum coatings.

While the preferred embodiment of the treatment device has been described with with specific reference to Mg—Zn—Ca—Mn alloys and/or BioMg 250™ alloy and the efficacy thereof, it is understood that other variations Mg alloys, including Mg—Zn—Ca alloy and other Mg alloys, or even pure Mg, may be provide in the form of some of the disclosed the treatment devices and implemented to control and treat cancerous tumors by the devices, system and methods disclosed herein.

1-7. (canceled)

8. Use of a Mg—Zn—Ca—Mn alloy in the manufacture of a medicament for the treatment of malignant tumors.

9. The use of a Mg—Zn—Ca—Mn alloy in the manufacture of a medicament according to claim 8, wherein the Mg—Zn—Ca—Mn alloy consists of, by weight 0.3 to 2.0% Zn, 0.2 to 1.0% Ca and 0.2 to 1.0% Mn, the remainder being Mg and inevitable impurities.

10. The use of a Mg—Zn—Ca—Mn alloy in the manufacture of a medicament according to claim 8, wherein the medicament is in the form of one of a wire, cylinder, mesh, sheet, screw, plate, coil, foil, powder or tube.

11. The use of a Mg—Zn—Ca—Mn alloy in the manufacture of a medicament according to claim 8, wherein the medicament is in the form of body defining a volume by inclusion of one or more recesses or bores provided therein or therethrough.

12. The use of a Mg—Zn—Ca—Mn alloy in the manufacture of a medicament according to claim 8, wherein one or more surfaces of the medicament are coated with anti-tumor agent agents.

13. The use of a Mg—Zn—Ca—Mn alloy in the manufacture of a medicament according to claim 12, wherein the anti-tumor agent is one of cisplatin or an alkylating agent containing platinum.

14. The use of a Mg—Zn—Ca—Mn alloy in the manufacture of a medicament according to claim 8, wherein one or more surfaces of the medicament are coated with a bioabsorption regulating coating, the bioabsorption regulating coating regulating initial absorption of the medicament.

15. The use of a Mg—Zn—Ca—Mn alloy in the manufacture of a medicament according to claim 8, wherein the medicament includes an anti-tumor agent.

16. The use of a Mg—Zn—Ca—Mn alloy in the manufacture of a medicament according to claim 8, wherein one or more surfaces of the medicament are coated with an X-Ray responding marker, such as platinum (Pt).

17. The use of a Mg—Zn—Ca—Mn alloy in the manufacture of a medicament according to claim 8, wherein the medicament is provided in the form of a pellet including at least one of spheres, cylinders, powder or wire formed of the Mg—Zn—Ca—Mn alloy and contained within a bioabsorbable membrane.

18-21. (canceled)

22. The use of a Mg—Zn—Ca—Mn alloy in the manufacture of a medicament according to claim 8, wherein the medicament includes radioactive seeds.

23. (canceled)

24. A method of treating a malignant tumor comprising administering to a patient in need there of a medicament including a Mg—Zn—Ca—Mn alloy.

25. The method of treating a malignant tumor according to claim 24, wherein the Mg—Zn—Ca—Mn alloy consists of, by weight 0.3 to 2.0% Zn, 0.2 to 1.0% Ca and 0.2 to 1.0% Mn, the remainder being Mg and inevitable impurities.

26. The method of treating a malignant tumor according to claim 24, wherein the malignant tumor is one of esophageal, gall bladder, osteosarcoma, bone metastasis, breast, testicular, ovarian, inguinal herniatric, bariatric, sphincter, lung, colorectal, and prostate cancers.

27-28. (canceled)

29. The method of treating a malignant tumor according to claim 24, wherein the medicament is in the form of at least one of a wire, cylinder, mesh, sheet, screw, plate, coil, foil, powder or tube.

30. The method of treating a malignant tumor according to claim 24, wherein medicament is one of placed, manipulation and shaped in the vicinity of the malignant tumor using laparoscopic surgery, manipulative human surgery and/or Artificial Intelligence robotic surgery.

31. The method of treating a malignant tumor according to claim 24, wherein the medicament is provided as an array of wire implants in or about the malignant tumor with inter-wire spacing of 2 to 5 mm.

32. The method of treating a malignant tumor according to claim 24, wherein the medicament is provided in combination with one of an external energy source of x-rays, eddy current, magnetic and/or microwave energy for enhancing the benefit of the medicament.

33. The method of treating a malignant tumor according to claim 24, wherein the medicament additionally includes chemotherapy agents.

34. The method of treating a malignant tumor according to claim 24, wherein the medicament is used in combination with one or more of an insertion device, a monitoring device and an external control device.

35. The method of treating a malignant tumor according to claim 34, wherein the insertion device a hypodermic needle.

36. The method of treating a malignant tumor according to claim 34, wherein the insertion device an introducer.

37. The method of treating a malignant tumor according to claim 24, wherein the medicament is placed in an area where the malignant tumor has been surgically removed.

38. (canceled)
